(19) United States
(12) Patent Application Publication
(10) Pub. No.: US 2005/0181027 A1
(43) Pub. Date: Aug. 18, 2005

(54) Chitosan-containing wound dressings

(76) Inventor: Horst Messinger, Aachen (DE)

Correspondence Address: COGNIS CORPORATION;PATENT DEPARTMENT, 300 BROOKSIDE AVENUE, AMBLER, PA, 19002, US

(21) Appl. No.: 11/055,911

(22) Filed: Feb. 11, 2005

(30) Foreign Application Priority Data
Feb. 13, 2004 (DE) 102004007115.2

(51) Int. Cl. A61K 9/70

(52) U.S. Cl. 424/445; 514/55

(57) ABSTRACT

The invention relates to antimicrobial wound dressings comprising (a) a biocompatible, permeable layer for application to the wound, then (b) a chitosan-containing interlayer in which chitosan is present in the form of granules, a film or a porous matrix and (c) at least one air- and oxygen-permeable layer acting as a supporting fabric and as a seal for the chitosan-containing interlayer. The embedding of the chitosan-containing layer guarantees the antimicrobial activity of the polymer without any swollen chitosan residues being left behind to contaminate the wound on removal of the dressing. The multilayer construction increases stability under mechanical stress.

### RELATED APPLICATIONS

This application claims priority from German application 102004007115.2 filed Feb. 13, 2004, the entire contents of which are incorporated by reference.

### FIELD OF THE INVENTION

This invention relates generally to the field of pharmacy and, more particularly, to dressing materials, more especially multilayer wound dressings, which contain chitosan in an interlayer as their antimicrobial active principle.

### BACKGROUND OF THE INVENTION

Bandages and dressings for treating wounds have to satisfy various requirements. Besides being easy to apply and handle and lending themselves to clean, painless removal, wound dressings also have to be able to adapt themselves flexibly to the wound and to provide protection against outside mechanical influences. Despite affording effective protection against environmental influences, wound dressings are expected to be sufficiently permeable to air and oxygen and also to water vapor.

The healing of wounds is influenced by a delicate moisture equilibrium. On the one hand, the secretion formed in the wound has to be removed, so that adequate absorbency of the final wound dressing is necessary for taking up and optionally removing moisture; on the other hand, a wound must not dry out because a certain moisture level is important to the granulation process. In addition, if a dressing were to dry out, the fresh granulation tissue formed would be destroyed on removal of the dressing through the sticking of the contact layer to the wound.

Above all, however, wound dressings are expected to be physiologically safe, kind to the skin and sterile. Accordingly, to reduce the risk of infection, an additional antimicrobial effect of wound dressings is desirable.

The use of chitosan in the medical and pharmaceutical field for its antimicrobial and anti-inflammatory properties is well-known. In particular, chitosan is frequently used for the antimicrobial finishing of wound dressings [Journal of Biomedical Materials Research, March, 2002, 59(3):438-449: Mi, F. L.; Wu, Y. B.; Shyu, S. S.; Shoung, J. Y.; Huang, Y. B., Tsai, Y. H., Hao, J. Y.; Control of wound infections using a bilayer chitosan wound dressing with sustainable antibiotic delivery.].

European Patent Application EP 1314410 A1 describes multilayer wound dressings comprising a moisture-absorbing layer of gel-forming fibers, including chitosan, and a transmission layer which is highly permeable to water vapor and which forms a barrier against outside influences. Unfortunately, chitosan does not have the mechanical strength to be effectively used for wound care without a supporting textile material. In addition, it swells considerably after wetting and the resulting gel lacks stability, particularly under mechanical load. Accordingly, a wound where the exudate caused the chitosan gel to swell has to be aftertreated by regular rinsing to remove the detached chitosan residues. This is time-consuming and expensive and also delays the wound healing process. To increase the dimensional stability of secretion-absorbing polymers, European patent application EP 0415183 A proposes the use of crosslinked hydrogels. However, the physiological compatibility of the alkoxysilyl compounds used as crosslinking agents has not yet been adequately investigated. In addition, crosslinking of the polymers leads to a deterioration in the antimicrobial properties of the chitosan. Experience has shown the mechanical properties of products consisting entirely of swollen chitosan to be unsatisfactory.

The chitosan films described in International patent application WO 95/01808 A1 are not particularly tear-resistant and, when dry, do not readily adapt themselves to intricate wound cavities. In this case, too, the chitosan film swells in a moist environment and forms a gel which, in wound dressings, forms residues that have to be regularly removed by rinsing of the wound.

Accordingly, the problem addressed by the present invention was to provide wound dressings with antimicrobial activity and adequate dimensional stability which could easily be adapted to shaped cavities and which would withstand mechanical stressing, would maintain the moisture equilibrium promoting the wound healing process and could be cleanly and painlessly removed.

### DESCRIPTION OF THE INVENTION

The present invention relates to wound dressings comprising
(a) a biocompatible, permeable layer for application to the wound, then
(b) a chitosan-containing interlayer in which chitosan is present in the form of granules, a film or a porous matrix and
(c) at least one air- and oxygen-permeable layer acting as a supporting fabric and as a seal for the chitosan-containing interlayer.

A flexible fabric protects the chitosan gel against direct contact with the wound tissue. A sandwich structure of the fabric is preferred, the inner layer consisting of flat or granulated chitosan. The exudate penetrates through the outer supporting fabric and causes the chitosan inside to swell, the chitosan also developing its antimicrobial effect. Active principles optionally added to the chitosan are continuously released and are able further to promote the wound healing process. Dressings are easy to change by changing the wound dressing; there is no need for rinsing or cleaning.

It has been found that the wound dressings according to the invention show good antimicrobial activity in two respects. On the one hand, antimicrobial activity has a positive effect in the wound dressing. Thus, although germ-containing wound secretion is effectively absorbed by the dressing, the antimicrobial finish still leads to a reduced germ load in the dressing, so that frequent dressing changes can be avoided. On the other hand, the antimicrobial properties also have an effect on the actual wound surface, because small quantities of dissolved chitosan molecules diffuse through the permeable membrane into the wound where they develop an antimicrobial effect and promote the wound healing process and tissue regeneration without leaving behind any gel-like residues that would have to be painfully removed after removal of the bandage.

In addition, the wound dressing according to the invention is distinguished by high dimensional stability, but still enables the surface of relatively deep cavities, for example in cases of decubitus, to be “lined” with the healing-promoting bandage. The absorbing effect of the chitosan gel keeps the wound free from exudate. Although the wound secretion passes quickly and lastingly through the biocompatible permeable layer into the chitosan-containing interlayer, the wound does not dry out by virtue of the swollen moisture-containing chitosan gel layer and the moisture equilibrium that promotes the wound healing process is maintained.

Chitosans

Chitosans are biopolymers which belong to the group of hydrocolloids. Chemically, they are partly deacetylated chitins differing in their molecular weights which contain the following—idealized—monomer unit: 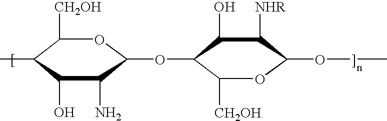
In contrast to most hydrocolloids, which are negatively charged at biological pH values, chitosans are cationic biopolymers under these conditions. The positively charged chitosans are capable of interacting with oppositely charged surfaces and are therefore used in cosmetic hair-care and body-care products and pharmaceutical preparations. Chitosans are produced from chitin, preferably from the shell residues of crustaceans which are available in large quantities as inexpensive raw materials. In a process described for the first time by Hackmann et al., the chitin is normally first deproteinized by addition of bases, demineralized by addition of mineral acids and, finally, deacetylated by addition of strong bases, the molecular weights being distributed over a broad spectrum. Corresponding processes are known, for example, from French patent application FR 2701266 A. Preferred types are those which are disclosed in German patent applications DE 4442987 A1 and DE 19537001 A1 (Henkel) and which have an average molecular weight of 10,000 to 5,000,000 dalton and more particularly 10,000 to 500,000 or 800,000 to 1,200,000 dalton and/or a Brookfield viscosity (1% by weight in glycolic acid) below 30,000 mPas, a degree of deacetylation of 80 to 88% and an ash content of less than 0.3% by weight. Chitosans with an average molecular weight of 10,000 to 5,000,000 dalton are normally used. A preferred embodiment is characterized by the use of chitosans with an average molecular weight of 30,000 to 1,000,000 dalton. Chitosans with a molecular weight of 40,000 to 500,000 dalton are also preferred, but chitosans with a molecular weight of 50,000 to 100,000 dalton are particularly preferred.

The chitosans obtainable in this way are chitosans of relatively high molecular weight. So-called oligoglucosamines, which are low molecular weight chitosans, are also particularly suitable. They are further degraded in a second step under the effect of acids and the degradation products, i.e. the oligoglucosamines, which now have a molecular weight of 500 to 5,000 and preferably in the range from 800 to 1,500, are subjected to membrane filtration to free them from impurities and especially salts. Besides antimicrobial activity, these oligochitosans also have a regenerative and growth-stimulating effect and show anti-inflammatory properties, so that they are particularly suitable for use in wound dressings.

Besides the chitosans as typical cationic biopolymers, derivatized chitosans where the cationic character is retained through the derivatization are also suitable for the purposes of the invention.

In order to prevent fine chitosan particles from penetrating through the porous wound contact layer (a), the chitosan is used in the form of granules, a porous matrix or film in the interlayer (b). Chitosan-containing films can be produced as described, for example, in WO 95/01808 A1.

Production of a Porous Matrix or Granules

To produce granules or a porous matrix, the chitosans are dissolved or suspended in aqueous mineral acids or aqueous organic carboxylic acids. The suspensions of the chitosans generally contain dissolved fractions of chitosans. Suitable mineral acids are hydrochloric acid, phosphoric acid, nitric acid and sulfuric acid; suitable organic carboxylic acids are formic acid, lactic acid, propionic acid, maleic acid, pyruvic acid, glycolic acid, succinic acid, acetic acid, citric acid, tartaric acid and adipic acid. Hydrochloric acid, lactic acid and glycolic acid are particularly preferred. The acid is used in the quantities required for partly or completely dissolving the chitosan. The quantities are normally 10⁻⁴ to 10⁻² mol acid groups per g chitosan and, more particularly, 1 to 3×10⁻³ mol acid groups per g chitosan.

The aqueous solution or suspension used is generally a 0.1 to 15% by weight aqueous solution or suspension, preferably a 0.5 to 10% by weight aqueous solution or suspension, more preferably a 1.0 to 5.0% by weight aqueous solution or suspension and most preferably a 1.5 to 2.5% by weight aqueous solution or suspension. If the suspension generally contains dissolved fractions, it can be of advantage to homogenize the suspension. The aqueous solution or homogenized suspension has a pH of generally 1.0 to 7.5 and more particularly 4.5 to 6.5.

A precipitant is generally used for the selective precipitation of the polymer from the solution or suspension. Precipitants suitable for the purposes of the invention are, in principle, any substances which increase the pH value of the aqueous solution or homogenized suspension. Aqueous solutions of carbonates, hydrogen carbonates, hydrogen phosphates and hydroxides of the alkali and alkaline earth metals, ammonia and organic nitrogen bases are suitable precipitants. Examples of suitable organic nitrogen bases include triethyl amine, triethanolamine or tetraalkyl ammonium hydroxides. The aqueous solutions of the precipitants are typically used in a concentration of 5 to 20% by weight and more particularly in a concentration of 7 to 16% by weight. In a preferred embodiment of the present invention, the precipitant used is an aqueous sodium hydrogen carbonate solution, more particularly a 7 to 16% by weight and preferably a 7 to 9% by weight aqueous sodium hydrogen carbonate solution. The treatment with the precipitant adjusts the pH of the aqueous solution or homogenized suspension of the biopolymers to a value of generally 5.0 to 14 and more particularly 7.0 to 8.5.

The product thus treated is then dried. Suitable drying methods are, for example, air drying, vacuum drying, more particularly at temperatures of 20 to 100° C. or above 100° C., and freeze drying.

The drying step may optionally be preceded by a freezing step. This is particularly advantageous in combination with freeze drying. The present invention includes the observation that the structure which is produced by precipitation of the fibers and fixed by the freezing step remains largely intact where drying is carried out in the form of freeze drying. Accordingly, the chitosan is obtained in a relatively stable form without the use of crosslinking agents. To this end, the suspension adjusted to the desired viscosity and mixed with precipitant is frozen at temperatures below its freezing point. The manner in which the freezing step is carried out has a major influence on the appearance and structure of the porous matrix formed after the freeze drying step. For example, the quicker the freezing step, the more finely porous and uniform the matrix formed after freeze drying.

If chitosan granules are used, the matrix produced after drying is size-reduced to the required particle size by any of the methods normally used for size-reducing solid materials. Granules may also be produced by pelletizing where the chitosan is used in conjunction with typical granulation auxiliaries. However, the purer chitosan granules produced with the fewest possible auxiliaries are preferred for wound dressings.

Production via freeze drying is preferred if other, temperature-sensitive active principles are to be incorporated in the chitosan granules or the chitosan matrix. These other auxiliaries and active principles may be added both before addition of the precipitant and also together with the precipitant. Where granules are used, the preparations are also charged with auxiliaries and active principles after the drying step. For example, cosmetic and pharmaceutical active principles or flavors are applied by special techniques to the final, dry preparation after the freeze drying step. To this end, the active principle is dissolved in a suitable solvent, applied to the sponge formed after freeze drying—which, in this embodiment, acts as a carrier material—and the solvent is then carefully removed. Suitable solvents are, for example, supercritical CO₂ or nonpolar or polar organic solvents such as, for example, hexane, ethanol or isopropanol. The dry application of fine-particle active principles to the granules is preferred.

Other Active Principles

Other active principles which accelerate the wound healing process and which may also be incorporated in the wound dressing according to the invention, preferably in the interlayer, are for example plant extracts, such as aloe vera, antibiotics, antiseptics, antimycotics, analgesics, antihistaminics, enzyme inhibitors, glucocorticoids, vitamins, virustatics, growth factors, steroids, enzymes or hormones. Where other active principles are used in the wound dressing, their delivery can be managed by controlled release. This can be done by a membrane which controls the release of active principles or by using selected molecular weights of the chitosan which then also acts as an active principle reservoir. Other polymers in the interlayer may optionally contribute to the release control function.

Construction of the Wound Dressing

The first layer, i.e. the biocompatible permeable layer (a) which comes into direct contact with the wound, is intended to separate the antimicrobial, wound-secretion-absorbing chitosan-containing layer from the wound. It must be permeable to the exudate, but should not absorb and store it because, otherwise, there would be a risk of microbial infestation. It ensures that the dimensional stability of the chitosan-containing interlayer remains intact, even after absorption of the wound secretion, and that there is no contamination by swollen particles in the wound and the dressing can be cleanly removed from the wound.

The permeable layer (a) may consist of nonwovens or porous membranes of biocompatible materials, such as silk, polyvinyl alcohol, polycarbonates, cellulose esters, such as cellulose acetate, cellulose nitrate, regenerated cellulose and polyolefins, such as polypropylene, polyethylene or copolymers of ethylene or propylene with butadiene. Polyester fibers, for example polyethylene terephthalate fibers, may also be used. Fibers consisting of two or more components, for example polyester/copolyester fibers or polypropylene/polyethylene fibers and polyamides, are also particularly suitable. Where nonwovens are used for layer (a), they may be produced by any of the methods for producing nonwovens known in the prior art as described, for example, in Ullmann's Encyclopedia of Industrial Chemistry, Vol. A 17, V C H Weinheim 1994, pages 572-581. Nonwovens produced either by the dry-laid process or by the spunbond process are preferred. The dry-laid process starts out from staple fibers which are normally first separated into individual fibers by carding and are then laid together to form the unstabilized nonwoven using an aerodynamic or hydrodynamic process. The unstabilized nonwoven is then heat-treated (“thermbonded”) to give the final nonwoven. To this end, the synthetic fibers are either heated to the extent that their surface melts and the individual fibers are joined together at their points of contact or the fibers are coated with an additive which melts during the heat treatment and thus bonds the individual fibers together. The individual bonds are fixed by cooling. Besides this process, any other processes used in the prior art for bonding nonwovens may of course also be used. By contrast, the spunbond process starts out from individual filaments formed by melt-spinning from extruded polymers which are forced under high pressure through spinning jets. The filaments issuing from the spinning jets are bundled, stretched and laid to form a nonwoven which is normally stabilized by thermobonding.

Where porous membranes are used, cellulose esters are preferably employed. To produce the membranes, the cellulose esters are dissolved in a readily volatile solvent and a small quantity of a relatively low-volatility nonsolvent is added to the resulting solution. The mixture formed is applied in a thin layer to an endless polished belt and aerated. The solvent evaporates and the relatively low-volatility nonsolvent increases in concentration until, finally, a gel of the macromolecular substance precipitates. The membrane film is obtained by drying, its pore structure being dependent upon the concentration of the starting solution and upon the evaporation conditions. In the production of porous polycarbonate membranes, a very thin nonporous film of polycarbonates is exposed to a neutron bombardment and then passed through an alkaline etching bath. At places where the neutrons have passed through, carbonate residues are loosened and are then dissolved out in the etching bath.

In the particular case of porous membranes, such as cellulose esters or polycarbonate membranes, a precisely defined pore size and porosity can be adjusted as required through the method of production, so that these materials are particularly suitable for wound dressings which contain additional active principles and which require controlled release.

The chitosan-containing interlayer (b), in which chitosan is present in the form of granules, a film or a porous matrix, may optionally contain other pharmaceutical active principles or plant extracts. These may be prepared by mixing the chitosan granules with other granules, powders or preparations or may be directly added during the production of the chitosan granules, the chitosan matrix or chitosan film.

Besides the chitosans, the interlayer may contain other swellable polymers. Since chitosan is a cationic biopolymer, the strength and dimensional stability of the interlayer may be further increased by combining the chitosan with anionic polymers and thus forming a relatively high viscosity, swelling gel. Corresponding polymers for the formation of so-called polyanion/polycation complexes are described in European patent application EP 1264014 A1.

Generally, however, the percentage content of chitosans in the interlayer should be at least 10% by weight and is preferably at least 50% by weight and, more particularly, at least 80% by weight, based on the material in the interlayer.

In a preferred embodiment, the chitosan-containing interlayer (b) is divided up into compartments. Dimensional stability is thus additionally increased and relatively deep cavities can be filled by the wound dressing without destroying the structure of the dressing. Simple and clean removal is also made possible in this way. The compartmental structure can be achieved by such techniques as darting or thermobonding. The quantity of chitosan delivered per unit area is thus always the same. The uniform chitosan delivery supported by the compartmental structure is particularly advantageous in the case of systems which contain additional active principles or which are intended for topical application.

The chitosan-containing interlayer is surrounded by at least one air- and oxygen-permeable layer (c) which acts as a supporting fabric and as a seal for the chitosan-containing interlayer. The layer (c) consists of a highly air-permeable fabric which effectively retains the chitosan granules and moisture. It should be impermeable to moisture, but optionally permeable to water vapor. This can also be achieved with a suitable combination of material layers. Suitable materials for this layer are, above all, polyesters, but also the other dermatologically compatible plastics already partly listed under the wound contact layer (a), such as polyvinyl chloride, ethylene/vinyl acetate copolymers, polyvinyl acetate, polyethylene, polypropylene, polyurethanes or cellulose derivatives and many others. In addition, this cover layer may be coated so that another layer is formed by vapor deposition of metals, more particularly aluminium, or other anti-diffusion additives, such as silicon dioxide for example.

### COMMERCIAL APPLICATIONS

The antimicrobial wound dressings according to the invention are distinguished by high dermatological compatibility and a high liquid uptake capacity without detriment to the necessary dimensional stability. Accordingly, the present invention relates to the use of the preparations according to the invention for wound treatment in the form of wound tampons, wound dressings, burn dressings, dressings which deliver active principles, nonwovens, transdermal therapeutic systems and dermal drug carriers. The preparations according to the invention may be charged with various pharmaceutical active principles and formulations intended for topical application. The wound dressings according to the invention may also be present in the form of a plaster with adhesive margins and thus provide for simple, convenient and quick handling. The dressing material may be sterilized in the usual way, preferably by ionizing radiation.

### EXAMPLES

### Example 1a

Production of Chitosan Granules

A suspension of 2 kg chitosan (Hydagen® CMFP, Henkel KGaA), 98 kg water and 0.346 kg L(+) lactic acid were homogenized in a colloid mill at a temperature of 40° C. until a viscosity of 23,000 mPas had been reached. The suspension was then cooled to 10° C. and degassed in vacuo. Quantities of 9 kg of the suspension were mixed for 2 minutes with 360 g of an aqueous solution of sodium hydrogen carbonate (=8.05% by weight of aqueous sodium hydrogen carbonate solution) and the resulting mixture was poured into molds. The layer thickness of the suspension in the mold was 22 mm. After standing for 3 h, the suspension was frozen and the frozen plates were then freeze-dried at 80° C./1 mbar.

The dried blocks were then size-reduced in a pin mill. A fraction with a mean particle size of 1 to 2 mm was removed by sieving.

### Example 1b

Chitosan granules were produced as described in Example 1a, the chitosan used being an oligoglucosamine with an average molecular weight of 500 which had been obtained by acidic degradation of chitosan (Hydagen® DCMF, Cognis Deutschland GmbH & Co. KG).

Production of the Wound Dressing

### Example 2a

The chitosan granules of Example 1 were distributed over a thermobonded polyamide nonwoven in such a way that 1±0.3 g granules were applied per square centimeter and were then covered with a breathable, water-impermeable polyester layer. The multilayer wound dressing was divided by darting into compartments with a width and length of ca. 1.3 cm.

### Example 2b

Example 2a was repeated with the granules of Example 1b.

### Example 3

Example 2a was repeated, the antibiotic silver sulfadiazine having been added to the granules of Example 1a by mixing the fine-particle powder with the chitosan granules in a quantity of 10 mg per g granules.

### Example 4

Example 2a was repeated, aloe vera (Alovera Konzentrat Pulver, Aloecare, 53501 Grafschaft) having been added to the granules of Example 1a by mixing the fine-particle powder with the chitosan granules in a quantity of 3% by weight, based on the quantity of granules.

The wound dressings described in the Examples were soaked with defined quantities of water to simulate exudate. In contrast to the chitosan films (Polymoist Marine, Cognis Deutschland GmbH & Co. KG) soaked with the same quantity of water per unit area, the described wound dressings retained their full mechanical stability and could be fixed in the required form to the human body without any chitosan passing through the biocompatible separating material, which could have contaminated a wound. By contrast, the pure Polymoist chitosan films in their swollen state were unable to withstand mechanical loads and could not be permanently fixed. The resulting chitosan fragments are difficult to remove from wounds and would be an obstacle to optimal healing.

1-9. (canceled)

10. A wound dressing comprising:
(a) a biocompatible, permeable layer for application to the wound;
(b) a chitosan-containing interlayer, wherein, the chitosan is present in the form of granules, a film or a porous matrix; and
(c) at least one air- and oxygen-permeable layer acting as a supporting fabric and as a seal for the chitosan-containing interlayer.

11. The wound dressing as claimed in claim 10, wherein, the chitosan-containing interlayer (b) is divided into compartments.

12. The wound dressing as claimed in claim 10, wherein, the chitosan-containing interlayer (b) contains additional active principles.

13. The wound dressing as claimed in claim 10, wherein, the chitosan-containing interlayer (b) comprises cationically derivatized chitosan.

14. The wound dressing as claimed in claim 10, wherein, the chitosan-containing interlayer (b) contains chitosan with an average molecular weight of 10,000 to 5,000,000.

15. The wound dressing as claimed in claim 10, wherein, the chitosan-containing interlayer (b) contains chitosan with an average molecular weight of 500 to 5,000.

16. The wound dressing as claimed in claim 10, wherein, the biocompatible permeable layer (a) which is adjacent to the wound controls release of an active component.

17. The wound dressing as claimed in claims 10, wherein, the wound dressing comprises a plaster with adhesive margins.

18. A method for treating and accelerating wound healing which comprises applying to the wound the wound dressing of claim 10.

19. The wound dressing of claim 11, wherein, the chitosan-containing interlayer (b) contains additional active principles.

20. The wound dressing as claimed in claim 11, wherein, the chitosan-containing interlayer (b) comprises cationically derivatized chitosans.

21. The wound dressing as claimed in claim 12, wherein, the chitosan-containing interlayer (b) comprises cationically derivatized chitosans.

22. The wound dressing as claimed in claim 12, wherein, the chitosan-containing interlayer (b) contains chitosan with an average molecular weight of 10,000 to 5,000,000.

23. The wound dressing as claimed in claim 12, wherein, the chitosan-containing interlayer (b) contains chitosan with an average molecular weight of 500 to 5,000.

24. The wound dressing as claimed in claim 12, wherein, the biocompatible permeable layer (a) which is adjacent to the wound controls release of an active component.

25. The wound dressing of claim 10, wherein, the chitosan is formed by a process comprising:
(a) dissolving chitosan in an aqueous mineral or organic carboxylic acid to form a mixture with a pH of from 1.0 to 7.5;
(b) increasing the pH of the mixture to a range of from 5 to 14 to form a mixture containing a precipitate containing chitosan;
(c) drying the mixture containing the precipitate to form a dried mixture comprising chitosan.

26. The wound dressing of claim 25, wherein, the dried mixture is subjected to size reduction and size selection.

27. The wound dressing of claim 25, wherein, the precipitate containing chitosan is dried by freeze drying.
